(19) United States
(12) Patent Application Publication
(10) Pub. No.: US 2024/0108887 A1
(43) Pub. Date: Apr. 4, 2024

(54) Reducing Electrosensation While Treating a Subject Using Alternating Electric Fields by Deactivating Selected Electrode Elements

(71) Applicant: Novocure GmbH, Root D4 (CH)

(72) Inventor: Yoram WASSERMAN, Haifa (IL)

(73) Assignee: Novocure GmbH, Root D4 (CH)

(21) Appl. No.: 18/374,080

(22) Filed: Sep. 28, 2023

Related U.S. Application Data
(60) Provisional application No. 63/411,939, filed on Sep. 30, 2022.

(51) Int. Cl. A61N 1/32; A61N 1/04

(52) U.S. Cl. CPC A61N 1/32; A61N 1/0476

(57) ABSTRACT

When arrays of electrode elements are used to apply alternating electric fields to a subject's body, the subject may experience electrosensation. This electrosensation can be ameliorated by selectively deactivating different electrode elements (or reducing the current that flows through different electrode elements) during respective different periods of time, and accepting feedback that indicates whether the electrosensation is occurring during each of those respective different periods of time. If deactivating a given one of the electrode elements (or reducing the current) ameliorates the electrosensation, the subject can be treated with alternating electric fields while the given electrode element is deactivated (or being driven with less current).

### CROSS REFERENCE TO RELATED APPLICATIONS

This application claims the benefit of U.S. Provisional Application 63/411,939, filed Sep. 30, 2022, is incorporated herein by reference in its entirety.

### BACKGROUND

Tumor Treating Fields (TTFields) therapy is a proven approach for treating tumors using alternating electric fields at frequencies between 50 kHz and 1 MHz (e.g., 150-200 kHz). In the prior art Optune® system, TTFields are delivered to patients via four transducer arrays that are placed on the patient's skin in close proximity to a tumor (e.g., on the front, back, left, and right sides of a person's head for a glioblastoma). The transducer arrays are arranged in two pairs, and each transducer array is connected via a multi-wire cable to an AC signal generator. The AC signal generator (a) sends an AC current through the anterior/posterior pair of transducer arrays for 1 second, which induces an electric field with a first direction through the tumor; then (b) sends an AC current through the left/right pair of arrays for 1 second, which induces an electric field with a second direction through the tumor; then repeats steps (a) and (b) for the duration of the treatment. Each transducer array includes a plurality (e.g., between 9 and 20) of electrode elements that are all wired together in parallel.

Alternating electric fields can also be used to treat medical conditions other than tumors. For example, as described in U.S. Pat. No. 10,967,167 (which is incorporated herein by reference in its entirety), alternating electric fields can be used to increase the permeability of the blood brain barrier (BBB) so that, e.g., chemotherapy drugs can reach the brain.

### SUMMARY OF THE INVENTION

One aspect of the invention is directed to a first method of ameliorating electrosensation in a subject that is being treated using alternating electric fields. The first method comprises selectively deactivating one or more different electrode elements during respective different periods of time while alternating electric fields are being applied; accepting feedback that indicates whether electrosensation is occurring during each of the respective different periods of time; and determining whether deactivating a given one or more of the electrode elements ameliorates the electrosensation.

Some instances of the first method further comprise treating the subject using alternating electric fields while the given one or more electrode elements is deactivated.

In some instances of the first method, the accepting of feedback is implemented by accepting input from the subject. In some instances of the first method, the accepting of feedback is implemented by processing electrical signals that represent activity of the subject's nerves from a set of ECAP electrodes. In some instances of the first method, the accepting of feedback is implemented by processing electrical signals that represent measurements of the subject's nerve or muscle activity. In some instances of the first method, the accepting of feedback is implemented by processing electrical signals that represent measurements of electromyography signals to measure muscle activity. In some instances of the first method, the accepting of feedback is implemented by processing electrical signals that represent measurements of an accelerometer to measure muscle activity.

Another aspect of the invention is directed to a second method of ameliorating electrosensation in a subject that is being treated using alternating electric fields. The second method comprises selectively reducing current that flows through one or more different electrode elements during respective different periods of time while alternating electric fields are being applied; accepting feedback that indicates whether electrosensation is occurring during each of the respective different periods of time; and determining whether reducing the current that flows through a given one or more of the electrode elements ameliorates the electrosensation.

Some instances of the second method further comprise treating the subject using alternating electric fields while the given one or more electrode elements is operating at a lower current than the other electrode elements.

In some instances of the second method, the accepting of feedback is implemented by accepting input from the subject. In some instances of the second method, the accepting of feedback is implemented by processing electrical signals that represent activity of the subject's nerves from a set of ECAP electrodes. In some instances of the second method, the accepting of feedback is implemented by processing electrical signals that represent measurements of the subject's nerve or muscle activity. In some instances of the second method, the accepting of feedback is implemented by processing electrical signals that represent measurements of electromyography signals to measure muscle activity. In some instances of the second method, the accepting of feedback is implemented by processing electrical signals that represent measurements of an accelerometer to measure muscle activity.

Another aspect of the invention is directed to a third method of applying electrical signals to a first set of at least four first electrode elements and a second set of at least four second electrode elements positioned on opposite sides of a target region of a subject's body. The third method comprises (a) applying an AC signal between the second set of at least four second electrode elements and a majority of the first electrode elements, wherein one or more different members of the first set of at least four first electrode elements are either not used or operate using a reduced current during respective different periods of time. The third method also comprises accepting first feedback indicating whether the subject is experiencing electrosensation or is about to experience electrosensation during the respective different periods of time; and determining, based at least in part on the accepted first feedback, whether an amelioration of electrosensation occurs when one or more given members of the first set of at least four first electrode elements is either not used or operates using the reduced current.

Some instances of the third method further comprise using the second set of at least four second electrode elements and all of the first electrode elements except for the one or more given members of the first set of at least four first electrode elements to apply alternating electric fields to the target region. Some instances of the third method further comprise applying alternating electric fields to the target region with the one or more given members of the first set of at least four first electrode elements operating at a lower current than other members of the first set.

In some instances of the third method, the accepting of the first feedback is implemented by accepting input from the subject. In some instances of the third method, the accepting of the first feedback is implemented by processing electrical signals that represent activity of the subject's nerves from a set of ECAP electrodes.

Some instances of the third method further comprise controlling an amplitude of the AC signal. Optionally, these instances may further comprise increasing the amplitude of the AC signal until the determining indicates that the subject is experiencing electrosensation or is about to experience electrosensation.

Some instances of the third method further comprise, prior to step (a), applying an AC signal between the second set of at least four second electrode elements and all of the first electrode elements during a first time period; and accepting feedback indicating whether the subject is experiencing electrosensation or is about to experience electrosensation during the first time period.

Some instances of the third method further comprise applying a second AC signal between the first set of at least four first electrode elements and a majority of the second electrode elements, wherein one or more different members of the second set of at least four second electrode elements are either not used or operate using a reduced current during respective different periods of time; accepting second feedback indicating whether the subject is experiencing electrosensation or is about to experience electrosensation during the applying of the second AC signal; and determining, based at least in part on the accepted second feedback, whether an amelioration of electrosensation occurs when one or more given members of the second set of at least four second electrode elements is either not used or operates using the reduced current. Optionally, these instances may further comprise using the first set of at least four first electrode elements and all of the second electrode elements except for the given member of the second set of at least four second electrode elements to apply alternating electric fields to the target region.

In some instances of the third method, the accepting of the first feedback is implemented by processing electrical signals that represent measurements of the subject's nerve or muscle activity. In some instances of the third method, the accepting of the first feedback is implemented by processing electrical signals that represent measurements of electromyography signals to measure muscle activity. In some instances of the third method, the accepting of the first feedback is implemented by processing electrical signals that represent measurements of an accelerometer to measure muscle activity.

In some instances of the third method, during the applying, at least two different members of the first set of at least four first electrode elements are either not used or operate using a reduced current during the respective different periods of time. In these instances, the determining comprises determining whether an amelioration of electrosensation occurs when at least two given members of the first set of at least four first electrode elements is either not used or operates using the reduced current.

Another aspect of the invention is directed to a first apparatus for applying electrical signals to a first set of at least four first electrode elements and a second set of at least four second electrode elements positioned on opposite sides of a subject's body. The first apparatus comprises an AC signal source, a first set of at least four first electrically controlled switches, and a controller. The AC signal source has at least one amplitude-control input. Each of the first switches is configured to, depending on a state of a respective first control input, either (i) close so that current can flow between the AC signal source and a respective first electrode element or (ii) open so that current does not flow between the AC signal source and the respective first electrode element. The controller is configured to control the state of the first control input of each of the first switches, and is further configured to (a) apply first control signals to the first control inputs that cause selected ones of the first switches to open at respective periods of time, (b) accept a plurality of first feedback signals indicating whether the subject is experiencing electrosensation or is about to experience electrosensation while the selected ones of the first switches is open at the respective periods of time, and (c) determine, based at least in part on the accepted plurality of first feedback signals, whether an amelioration of electrosensation occurs when a given one of the first switches is open.

In some embodiments of the first apparatus, the controller is further configured to control the application of the electrical signals to the first set of at least four first electrode elements and the second set of at least four second electrode elements so that while the subject is being treated with alternating electric fields, the given one of the first switches is open.

Some embodiments of the first apparatus further comprise a user interface, wherein the user interface is configured to, based on inputs received from the subject, generate the plurality of first feedback signals. Some embodiments of the first apparatus further comprise an ECAP measurement system configured to accept signals that represent nerve activity from a set of ECAP electrodes, wherein the ECAP measurement system generates the plurality of first feedback signals.

In some embodiments of the first apparatus, the controller is further configured to control the amplitude-control input. Optionally, in these embodiments, the controller may be further configured to, prior to step (a), apply first control signals to the first control inputs that cause all the first switches to close, control the amplitude-control input so that the amplitude increases, and accept a third signal indicating whether the subject is experiencing electrosensation or is about to experience electrosensation.

Some embodiments of the first apparatus further comprise a second set of at least four second electrically controlled switches, wherein each of the second switches is configured to, depending on a state of a respective second control input, either (i) close so that current can flow between the AC signal source and a respective second electrode element or (ii) open so that current does not flow between the AC signal source and the respective second electrode element. In these embodiments, the controller is further configured to control the state of the second control input of each of the second switches, and the controller is further configured to (x) apply second control signals to the second control inputs that cause selected ones of the second switches to open at respective periods of time, (y) accept a plurality of second feedback signals indicating whether the subject is experiencing electrosensation or is about to experience electrosensation while the selected ones of the second switches is open at the respective periods of time, and (z) determine, based at least in part on the accepted plurality of second feedback signals, whether an amelioration of electrosensation occurs when a given one of the second switches is open.

Optionally, in the embodiments of the first apparatus described in the previous paragraph, the controller is further configured to control the application of the electrical signals to the first set of at least four first electrode elements and the second set of at least four second electrode elements so that while the subject is being treated with alternating electric fields, the given one of the second switches is open. Optionally, the embodiments described in the previous paragraph may further comprise a user interface, wherein the user interface is configured to, based on inputs received from the subject, generate the plurality of first feedback signals and the plurality of second feedback signals. Optionally, the embodiments described in the previous paragraph may further comprise an ECAP measurement system configured to accept signals that represent nerve activity from a set of ECAP electrodes, wherein the ECAP measurement system generates the plurality of first feedback signals and the plurality of second feedback signals.

Some embodiments of the first apparatus further comprise a second set of at least four second electrically controlled switches, wherein each of the second switches is configured to, depending on a state of a respective second control input, either (i) close so that current can flow between the AC signal source and a respective second electrode element or (ii) open so that current does not flow between the AC signal source and the respective second electrode element. In these embodiments, the controller is further configured to control the state of the second control input of each of the second switches, and the controller is further configured to (x) apply second control signals to the second control inputs that cause selected ones of the second switches to open at respective periods of time, (y) accept a plurality of second feedback signals indicating whether the subject is experiencing electrosensation or is about to experience electrosensation while the selected ones of the second switches is open at the respective periods of time, and (z) determine, based at least in part on the accepted plurality of second feedback signals, whether an amelioration of electrosensation occurs when a given one of the second switches is open. And the controller is further configured to control the amplitude-control input.

Optionally, in the embodiments of the first apparatus described in the previous paragraph, the controller is further configured to, prior to step (a), apply first control signals to the first control inputs that cause all the first switches to close, control the amplitude-control input so that the amplitude increases, and accept a third signal indicating whether the subject is experiencing electrosensation or is about to experience electrosensation.

### BRIEF DESCRIPTION OF THE DRAWINGS

FIG. 1 depicts how transducer arrays are positioned on a subject's head for treating glioblastoma using alternating electric fields.

FIG. 2 is a block diagram of an embodiment for treating a subject using alternating electric fields with one or more electrode elements deactivated to ameliorate electrosensation.

FIG. 3 is a flowchart of an approach for determining which transducer array is contributing the most to the electrosensation.

FIG. 4 is a flowchart of an approach for determining which electrode element within a given transducer array is contributing the most to the electrosensation.

FIG. 5 is a block diagram of another embodiment for treating a subject using alternating electric fields with one or more electrode elements deactivated to ameliorate electrosensation.

Various embodiments are described in detail below with reference to the accompanying drawings, wherein like reference numerals represent like elements.

### DESCRIPTION OF THE PREFERRED EMBODIMENTS

When treating a subject using alternating electric fields, higher amplitudes are strongly associated with higher efficacy of treatment. However, as the amplitude of the alternating electric field increases, and/or as the frequency of the alternating electric field decreases (e.g., to the vicinity of 100 kHz), some subjects experience an electrosensation effect when the alternating electric field switches direction. This electrosensation could be, for example, a vibratory sensation, paresthesia, and/or a twitching or contraction sensation of muscle fibers, or a flicker of light in the eyes (phosphene). And these sensations may discourage some subjects from continuing their treatment using alternating electric fields. The electrosensation is believed to originate from interactions between the alternating electric fields and nerve cells or fibers (i.e., neurons or axons) that are positioned near or adjacent to the transducer arrays. Disclosed herein are apparatuses and methods for reducing electrosensation while treating a subject using alternating electric fields by deactivating one or more electrode elements.

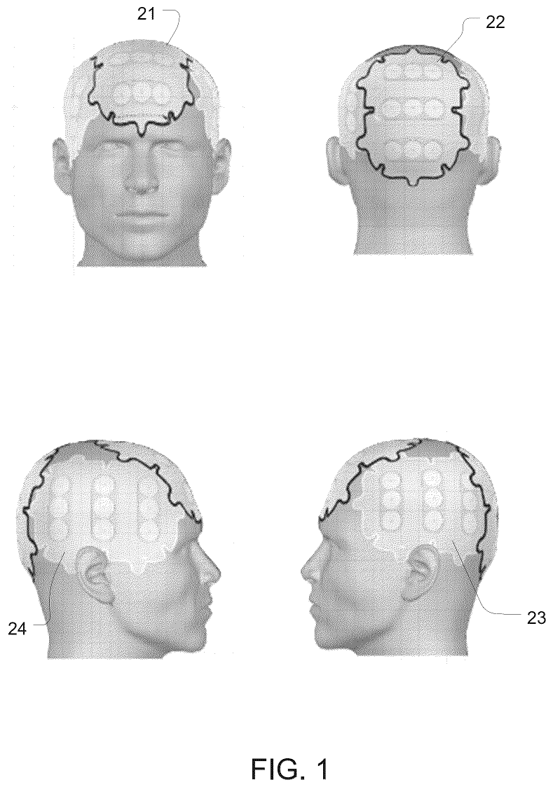

FIG. 1 illustrates an example in which four transducer arrays 21, 22, 23, and 24 are respectively positioned on the anterior, posterior, left, and right sides of a subject's head. As the amplitude of the alternating electric field increases and/or as the frequency of the alternating electric field decreases, the subject may begin to experience electrosensation beneath or near one or more of the transducer arrays 21-24.

It is possible that the onset of electrosensation will occur simultaneously beneath/near all four of the transducer arrays 21-24 as the amplitude of the alternating electric field increases and/or as the frequency decreases. But it is also possible for the onset of electrosensation to occur beneath only one, two, or three of those transducer arrays. Assume, for purposes of discussion, that when the amplitude of the alternating electric field reaches a given value, a given subject begins to experience electrosensation only beneath the left transducer array 23. This electrosensation will limit the amplitude of the alternating electric field that can comfortably be applied to the given subject, which in turn limits the efficacy of the treatment.

In the illustrated example, each of the transducer arrays 21-24 (including the left transducer array 23) includes nine electrode elements. But in alternative examples, each transducer array 21-24 could include a different number of electrode elements (e.g., between 4 and 50 electrode elements). Each transducer array 21-24 is similar in many respects to the prior art Optune® transducer arrays. But unlike the Optune® transducer arrays, the electrode elements within any given transducer array in FIG. 1 are not all wired together in parallel. Instead, each electrode element within any given transducer array 21-24 is provided with an independent conductor, so that each electrode element within any given transducer array can be activated or deactivated independently. One example of a suitable approach for implementing transducer arrays with electrode elements that can be activated or deactivated independently is disclosed in U.S. Pat. No. 11,395,916 (Wasserman at al., hereinafter referred to as “the '916 patent”), which is incorporated herein by reference in its entirety.

Due to either the layout of nerve fibers in the subject's body, the presence of sweat, or other factors, it is possible that the electrosensation may be attributable to only a small number (e.g., one or two) of the nine electrode elements within a given transducer array. In this situation, the electrosensation can be ameliorated by selectively deactivating one or more different electrode elements during respective different periods of time while alternating electric fields are being applied; accepting feedback that indicates whether electrosensation is occurring during each of the respective different periods of time; and determining whether deactivating a given one (or more than one) of the electrode elements ameliorates the electrosensation. The subject is then treated using alternating electric fields while the given electrode element is deactivated. This can make it possible to increase the amplitude of the alternating electric fields without causing discomfort to the subject.

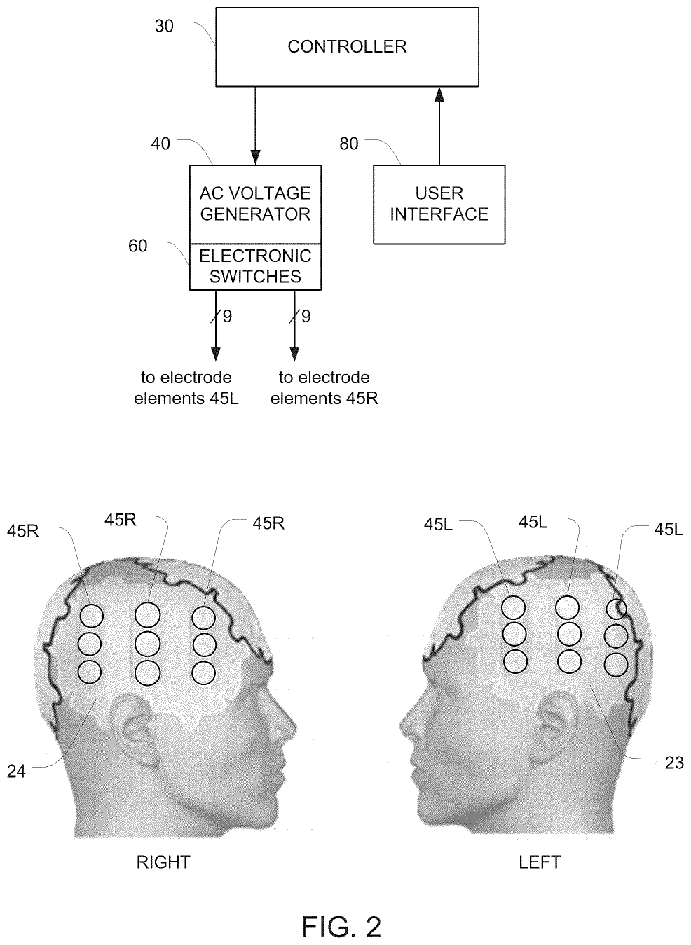

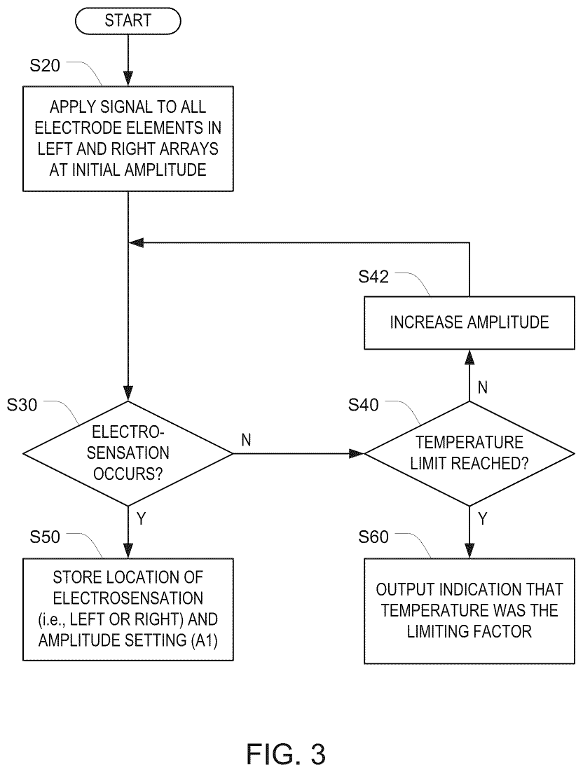

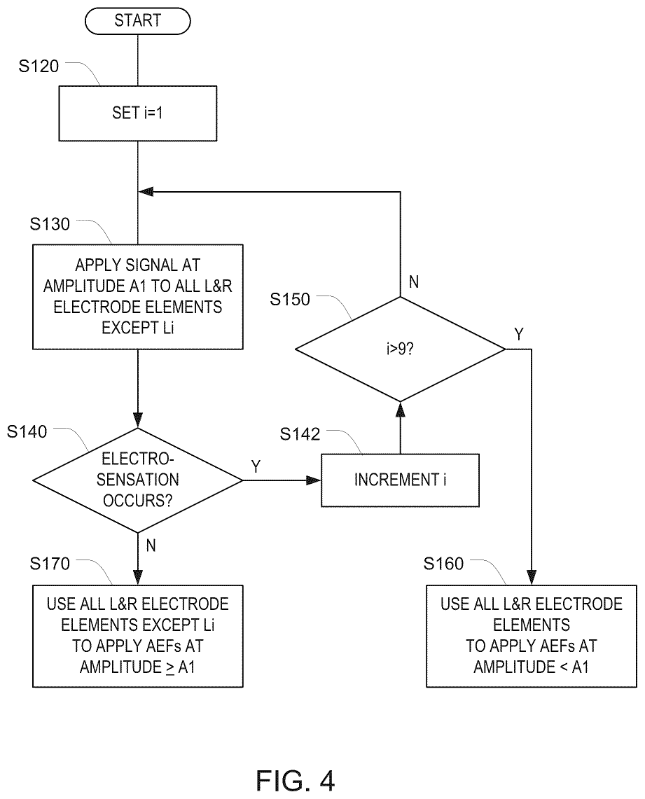

FIGS. 2-4 depict a first approach for determining which electrode elements within the transducer arrays are responsible for the electrosensation for a given subject, and subsequently deactivating those electrode elements when the transducer arrays are used to treat the subject with an alternating electric fields therapy (e.g., TTFields). More specifically, FIG. 2 is a block diagram of one embodiment for treating a subject using alternating electric fields with one or more electrode elements deactivated to ameliorate electrosensation; FIG. 3 is a flowchart of an approach for determining which transducer array is contributing the most to the electrosensation; and FIG. 4 is a flowchart of an approach for determining which electrode element within a given transducer array is contributing the most to the electrosensation.

FIG. 2 depicts an apparatus for treating a target region of a subject's body with an alternating electric field that avoids or ameliorates electrosensation based on feedback from the user that is accepted via a user interface 80. The FIG. 2 embodiment includes an AC voltage generator 40 that generates AC outputs at a frequency between 50 kHz and 1 MHz (e.g., 50-500 kHz, 75-300 kHz, or 150-250 kHz). The AC voltage generator 40 has at least one control input which may be used, for example, to control the output amplitude of the AC voltage generator 40. The frequency of the AC voltage generator 40 will depend on the type of treatment. For example, to treat a tumor using TTFields, the frequency could be between 150 and 200 kHz. Alternatively, to increase the permeability of a subject's blood-brain barrier, the frequency could be between 50 kHz and 200 kHz (e.g., 100 kHz).

In the example depicted in FIG. 2, a transducer array 23 that includes a set of first electrode elements 45L is positioned on the subject's body (e.g., on shaved skin) to the left of a target region, and a transducer array 24 that includes a set of second electrode elements 45R is positioned on the subject's body to the right of the target region. In alternative embodiments, the first and second sets of electrode elements 45L/45R could be implanted in the subject's body (e.g., just beneath the skin) to the left and right of the target region, respectively. When the AC voltage generator 40 applies a voltage between the electrode elements 45L and the electrode elements 45R, an alternating electric field is induced through the target region with field lines that run generally from right to left. The frequency of the alternating electric field will match the frequency of the AC voltage generator 40. The electrode elements 45L/45R can be capacitively-coupled electrode elements or conductive electrode elements.

Notably, the electrode elements within any given transducer array 23-24 are not all wired together in parallel. Instead, each electrode element within any given transducer array 23-24 is provided with an independent conductor, so that each electrode element within any given transducer array can be activated or deactivated independently. This may be accomplished using a bank of electronic switches 60 that includes a dedicated switch for each electrode element 45R in the right transducer array 24, and also includes a dedicated switch for each electrode element 45L in the left transducer array 23 (e.g., as described in the '916 patent). Thus, in the example depicted in FIG. 2 (where the left and right transducer arrays 23, 24 each include nine electrode elements), the bank of electronic switches 60 will have 9×2=18 switches.

The AC voltage generator 40 has one output pin for feeding the left electrode elements 45L, and another output pin for feeding the right electrode elements 45R. Nine of the switches in the bank 60 either (a) allow the signal from the left output pin of the AC voltage generator 40 to reach a respective one of the electrode elements 45L in the transducer array 23 or (b) prevent the signal from the left output pin of the AC voltage generator 40 from reaching that respective electrode element, depending on the state of control signals that arrive from the controller 30. And an additional nine switches in the bank 60 either (a) allow the signal from the right output pin of the AC voltage generator 40 to reach a respective one of the electrode elements 45R in the transducer array 24 or (b) prevent the signal from the right output pin of the AC voltage generator 40 from reaching that respective electrode element, depending on the state of control signals that arrives from the controller 30. This allows the controller 30 to either activate or deactivate each of the electrode elements 45L in the left transducer array 23 independently, and also allows the controller 30 to either activate or deactivate each of the electrode elements 45R in the right transducer array 24 independently.

A user interface 80 accepts inputs from a user indicating whether the subject is experiencing electrosensation. It may be implemented, for example, using a touchscreen, a keyboard, dedicated pushbuttons, voice recognition, or any of a variety of other approaches that will be apparent to persons skilled in the relevant arts.

Assume that we have a situation in which a subject is experiencing electrosensation. The flowcharts in FIGS. 3 and 4 may be used to identify which electrode element in which transducer array is contributing the most to the electrosensation. And once this information is ascertained, the controller 30 can deactivate the identified electrode element. Treatment of the patient with alternating electric fields (e.g., TTFields) can then proceed.

Beginning with S20 in the FIG. 3 flowchart, the controller 30 sends control signals to the bank of switches 60 so that the signal from the left terminal of the AC voltage generator 40 is applied to all of the electrode elements 45L in the left transducer array 23, and so that the signal from the right terminal of the AC voltage generator 40 is applied to all of the electrode elements 45R in the right transducer array 24. The controller 30 also sends control signals to the AC voltage generator to control the amplitude of the AC voltage.

Next, in S30, the controller 30 determines whether electrosensation is occurring. This determination is based on inputs received from the user interface 80. If electrosensation is not occurring, processing proceeds to S40 where the controller determines whether a temperature limit (e.g., 41° C.) has been reached. If the temperature limit has not been reached, processing proceeds to S42, where the controller 30 issues commands to the AC voltage generator to increase the amplitude. Processing then returns to S30, and the S30/S40/S42 loop will continue until either electrosensation occurs or the temperature limit has been reached.

If the temperature limit was reached (which can be ascertained, for example, based on inputs from temperature sensors incorporated into the transducer arrays) processing proceeds to S60, where the controller 30 outputs an indication that temperature was the limiting factor.

If electrosensation occurs (which is ascertained based on inputs from the user interface 80 that inform the controller 30 indicating which transducer array 23/24 caused the electrosensation), processing proceeds to S50, where the controller 30 stores an indication of which transducer array (i.e., the left array 23 or the right array 24) was responsible for the electrosensation, and also the amplitude setting A1 of the AC signal generator 40 at which the electrosensation began.

Once the controller 30 has identified which transducer array is responsible for the electrosensation (i.e., the left array 23 or the right array 24), the controller 30 orchestrates the application of electrical signals to a first set of at least four first electrode elements and a second set of at least four second electrode elements positioned on opposite sides of a subject's body. (Note that in this paragraph and in the paragraphs that discuss FIG. 4 below, the electrode elements on the side that was responsible for the electrosensation are referred to as the first set of first electrode elements, and the electrode elements on the opposite side are referred to as the second set of second electrode elements.) The controller 30 does this by setting the switches in bank 60 so that an AC signal is applied between the second set of at least four second electrode elements and a majority of the first electrode elements, wherein different members of the first set of at least four first electrode elements are not used during respective different periods of time; accepting first feedback (via the user interface 80) indicating whether the subject is experiencing electrosensation or is about to experience electrosensation during the respective different periods of time; and determining, based at least in part on the accepted first feedback, whether an amelioration of electrosensation occurs when a given member of the first set of at least four first electrode elements is not used.

One example of how the controller 30 can accomplish the steps set forth in the previous paragraph is to implement the flowchart of FIG. 4 to determine if a specific electrode element in the identified transducer array is contributing to the electrosensation more than the other electrode elements in that transducer array. More specifically, in S120, a loop is initialized, and in S130-150 a loop is implemented to set up the switches in the bank 60 so that AC signals at an amplitude A1 are applied to all but one of the first electrode elements, and feedback is accepted from the user via the user interface 80 to see if electrosensation is occurring. In each pass through the loop, a different one of the first electrode elements is deactivated. If, in a given pass through the loop, electrosensation does not occur, processing jumps from S140 to S170, where the second set of second electrode elements and all of the first electrode elements except for the given one of the first set of electrode elements are used to apply alternating electric fields (“AEFs”) to a target region in the subject's body. In this way, the electrode element that provided the biggest contribution to electrosensation is deactivated, and the amplitude of the AC signals generated by the AC voltage generator 40 can be increased to a level that is above A1.

If none of the passes through the loop were free from electrosensation (as reported via the user interface 80), then processing jumps from S150 to S160 where all the electrode elements in the first and second sets of electrode elements are used to apply alternating electric fields to the subject's body and at an amplitude that is below A1.

Note that instead of using the steps shown in FIG. 3 to isolate the electrosensation problem to a particular transducer array, followed by the steps shown in FIG. 4 to isolate the electrosensation problem to a given electrode element within the particular transducer array by deactivating one electrode element at a time until the electrosensation goes away, an alternative approach may be used. In this alternative approach, the steps shown in FIG. 3 (which narrow down the electrosensation problem to a specific transducer array) are omitted. Instead, the process starts by performing the steps shown in FIG. 4 for a given one of the transducer arrays by deactivating one electrode element at a time to ascertain if the electrosensation goes away when one of the electrode elements is deactivated. If none of the deactivations for the given one of the transducer arrays eliminates the electrosensation, the controller 30 performs the steps shown in FIG. 4 on the next transducer array, and deactivates one electrode element at a time to ascertain if the electrosensation goes away when one of the electrode elements in the second transducer array is deactivated. If a deactivation of an electrode element on the second transducer array causes the electrosensation to go away, then the controller 30 has identified the electrode element that is causing the problem, and can proceed with treating the subject while that electrode element is deactivated.

The operation of the FIG. 2 embodiment is not limited to the specific situation described above in connection with FIGS. 3 and 4 in which only a single electrode element on any given transducer array is responsible for the electrosensation. To the contrary, two or more electrode elements on a given transducer array may be responsible for the electrosensation. In this situation, more than one electrode element can be switched off at respective different periods of time, and feedback is accepted indicating whether the subject is experiencing electrosensation or is about to experience electrosensation during the respective different periods of time. Finally, the system determines, based at least in part on the accepted feedback, whether an amelioration of electrosensation occurs when the two or more electrode elements are not used.

Returning to FIG. 2, instead of using the bank of switches 60 to individually switch the current on and off through each of the electrode elements 45L, 45R, a bank of circuits that can individually impede the current that flows through each of the electrode elements may be used. When any of the circuits in the bank is activated, it reduces the current that flows through a respective one of the electrode elements 45 instead of shutting the current off completely. In other respects, these embodiments are similar to the embodiments described above in connection with FIGS. 2-4.

Additional situations can be envisioned in which two or more electrode elements on a given transducer array will cause electrosensation. One example is when the electrode elements are configured in groups, and the groups are arranged so that all of the elements within any given group are switched on or off together. For example, a 3×3 array of electrode elements may be arranged into three groups, with three electrode elements in each group. In this situation, instead of switching off each electrode element individually to ascertain whether the subject is experiencing electrosensation (or is about to experience electrosensation), as described above in connection with FIG. 4, each group of electrode elements is switched off together to ascertain whether the subject is experiencing electrosensation when that group is switched off. If it turns out that any group is responsible for the electrosensation, that group can be disabled or the current to that group can be reduced in order to ameliorate the electrosensation.

In another situation, the electrode elements may be arranged in groups of two or more closely-spaced electrode elements, where the individual elements within any group are either switched on or off to deliver different levels of current to a specific area on the subject's body. For example, 18 electrode elements could be arranged as a 3×3 array of electrode element pairs. When both elements within any given pair are on, a given level of current is delivered to the spot beneath that pair. But when only one of the elements within any given pair is on, only 50% of the given level of current is delivered to that spot. Here again, the current that is delivered to each pair of electrode elements can be reduced in turn to ascertain whether the subject is experiencing electrosensation (or is about to experience electrosensation). If it turns out that any group is responsible for the electrosensation, that group can be operated at a reduced current in order to ameliorate the electrosensation.

In many anatomic locations, it is preferable to use an electric field whose orientation alternates between different directions. In these locations, additional transducer arrays, each of which includes a set of at least four electrode elements 45 (not shown in FIG. 2) may be positioned on other sides (e.g., anterior and posterior) of the target region. In these embodiments, the AC voltage generator 40 is preferably configured to repeatedly alternate between (a) applying a voltage between the left and right electrode elements 45L/45R, and (b) applying a voltage between the anterior and posterior electrode elements. The AC voltage generator 40 can switch between these two states every 1 second, or at a different interval (e.g., between 50 ms and 10 s). The orientation of the electric field in these embodiments will therefore repeatedly alternate back and forth between the left/right and anterior/posterior directions.

In these bidirectional embodiments, the bank of electronic switches 60 includes a dedicated switch for each electrode element in each additional array. Thus, in an example case where each transducer array includes nine electrode elements, the bank of electronic switches 60 will have 9×4=36 switches. This allows the controller 30 to either activate or deactivate each of the electrode elements in the additional transducer arrays independently. The additional transducer arrays (i.e., the anterior and posterior transducer arrays 21-22) are handled in a manner similar to the left and right transducer arrays 23-24 described above.

In the embodiments described above in connection with FIGS. 2-4, the controller 30 determines that electrosensation is occurring (in S30 and S140) based on feedback from the subject that is received via the user interface 80. But in alternative embodiments, instead of relying on a user interface to inform the system that electrosensation is occurring, the system can automatically detect that the subject is experiencing electrosensation (or is about to experience electrosensation) using hardware that measures an electrically evoked compound action potential (ECAP).

During certain types of electrical stimulation of biological tissue, the electrically evoked compound action potential (ECAP) represents the approximately synchronous firing of a population of electrically stimulated nerve fibers. Upon the application of an electrical signal of sufficient energy to activate nerve fibers, fibers of different diameters and in different locations are activated at roughly the same time (e.g., within fractions of milliseconds) and their action potentials (APs) propagate at different velocities to the vicinity of a recording electrode. Further, different nerve fibers of different diameters, which have different activation thresholds and conduction velocities, convey different signals, e.g., of types of sensation (vibration, temperatures, hair movement, muscle contraction, joint position, etc.).

It turns out that the ECAP associated with electrosensation can be measured using a set of electrodes positioned on a subject's skin. These electrodes detect the compounded sum of the individual APs arriving at approximately the same time, which appear as a curve of a given amplitude and duration. Signals from these electrodes can be used to detect or predict whether the subject is probably experiencing electrosensation or is about to experience electrosensation.

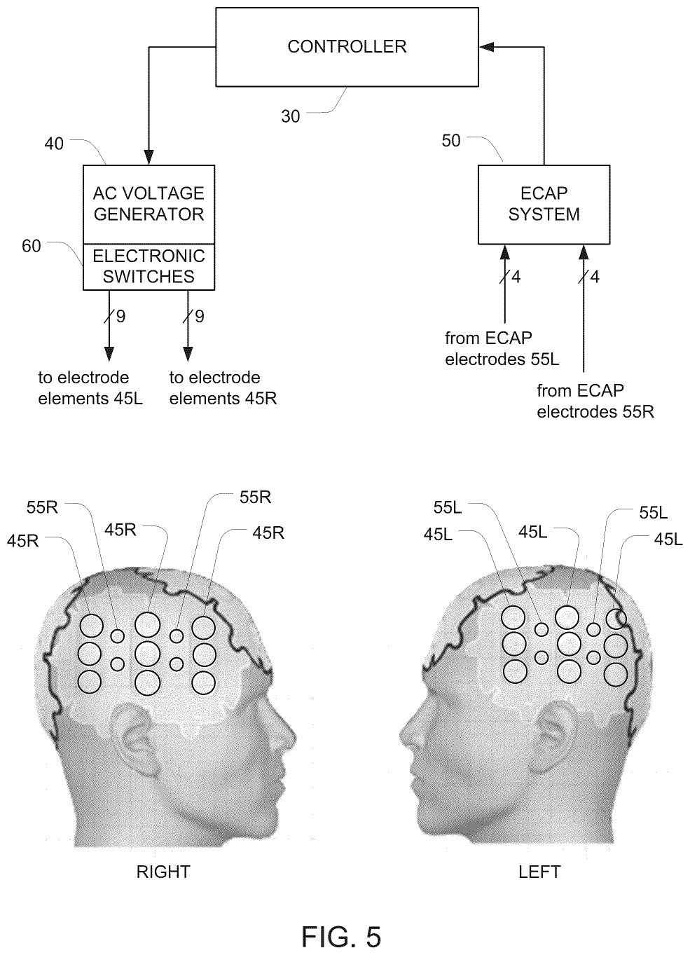

FIG. 5 depicts an embodiment that relies on ECAP signals that are received using such electrodes to detect or predict whether the subject is probably experiencing electrosensation or is about to experience electrosensation. In these embodiments, the ECAP signals provide feedback to the controller 30 (instead of the user interface 80 that is used in the FIG. 2 embodiment).

The AC voltage generator 40, the bank of switches 60, and the electrode elements 45L, 45R in this FIG. 5 embodiment are similar to the corresponding elements in the FIG. 2 embodiment described above. And the operation of the controller 30 is also similar to the operation of the controller in the FIG. 2 embodiment, with an important exception. More specifically, instead of the FIG. 2 approach of accepting feedback indicating that the subject is experiencing electrosensation from a user interface, in this FIG. 5 embodiment, the controller 30 accepts feedback indicating whether the subject is experiencing electrosensation or is about to experience electrosensation from an ECAP System 50.

In this FIG. 5 embodiment, in addition to the electrode elements 45L/45R which are used to induce the alternating electric field in the target region, independent sets of electrodes 55L, 55R are also provided to determine whether the subject is probably experiencing electrosensation or is about to experience electrosensation. More specifically, a first set of ECAP electrodes 55L configured for picking up ECAP signals is positioned near the set of first electrodes 45L, and a second set of ECAP electrodes 55R configured for picking up ECAP signals is positioned near the set of second electrodes 45R. The first and second sets of ECAP electrodes 55L, 55R positioned on the left and right sides, respectively, could each be a passive array of electrodes.

Signals from these ECAP electrodes 55L, 55R (which can be, e.g., on the order of 0.2-2 mV) are accepted by the ECAP measurement system 50, and the ECAP measurement system 50 measures the ECAP on the left and right sides of the subject's body based on signals that arrive from the ECAP electrodes 55L, 55R positioned on the left and right sides, respectively. The ECAP measurement system 50 processes those signals (e.g., using an amplifier and an analog to digital converter) and forwards the resulting data to the controller 30. In this way, the ECAP that is generated by each side of the subject's body in response to the application of the alternating electric field is measured.

Because the ECAP associated with electrosensation is measured using the ECAP electrodes 55L, 55R and the ECAP measurement system 50, and those measurements are reported to the controller 30, the controller 30 can determine whether the subject is probably experiencing electrosensation or is about to experience electrosensation. The controller 30 in this FIG. 5 embodiment can therefore implement steps that are similar to those described above in connection with FIGS. 3-4, except that instead of receiving feedback from a user interface that electrosensation is occurring (in S30 and S140), the controller 30 in this FIG. 5 embodiment receives feedback from the ECAP system 50 indicating whether the subject is experiencing electrosensation or is about to experience electrosensation.

As explained above, in some anatomic locations it is preferable to use an electric field whose orientation alternates between different directions. In these locations, additional transducer arrays positioned on other sides (e.g., anterior and posterior) of the target region may be provided. In this situation, each set of electrode elements 45 preferably has its own associated set of ECAP electrodes 55x, which is used to determine whether the subject is probably experiencing electrosensation or is about to experience electrosensation. The controller 30 responds to signals arriving from these additional sets of ECAP electrodes 55x (preprocessed by the ECAP system 50) as described above for the signals that arrive from the left and right sets of ECAP electrodes 55L, 55R (which are also preprocessed by the ECAP system 50).

The FIG. 5 embodiment relies on a passive array of ECAP electrodes to measure nerve activity, based on the theory that nerve activity can be used to determine that electrosensation is occurring or imminent. But a variety of alternative approaches to automatically determine that electrosensation is occurring or imminent may be used instead of the ECAP-based techniques described above. One example of an alternative approach uses electromyography signals to measure muscle activity, based on the theory that muscle activity (e.g., twitching) can be an indication that electrosensation is occurring. In these embodiments, the electromyography (EMG) signals are obtained using a set of EMG electrodes, pre-processed by an EMG system, and forwarded to a controller (which is similar to the controller 30 described above, but programmed to interpret EMG signals instead of ECAP signals). Another example of an alternative approach uses a mechanical sensor (e.g., an accelerometer) to measure muscle activity, based on the theory that muscle activity (e.g., twitching) can be an indication that electrosensation is occurring. In these embodiments, the vibration or acceleration signals are captured using the mechanical sensor, pre-processed by an appropriate front end, and forwarded to a controller (which is similar to the controller 30 described above, but programmed to interpret mechanical events instead of ECAP signals). Other approaches based on measured nerve or muscle activity can also be used.

### ILLUSTRATIVE EMBODIMENTS

Embodiment 1 is a method of ameliorating electrosensation in a subject that is being treated using alternating electric fields, the method comprising: selectively deactivating one or more different electrode elements during respective different periods of time while alternating electric fields are being applied; accepting feedback that indicates whether electrosensation is occurring during each of the respective different periods of time; and determining whether deactivating a given one or more of the electrode elements ameliorates the electrosensation.

Embodiment 2 is the method of embodiment 1, further comprising treating the subject using alternating electric fields while the given one or more electrode elements is deactivated.

Embodiment 3 is the method of embodiment 1, wherein the accepting of feedback is implemented by accepting input from the subject.

Embodiment 4 is the method of embodiment 1, wherein the accepting of feedback is implemented by processing electrical signals that represent activity of the subject's nerves from a set of ECAP electrodes.

Embodiment 5 is the method of embodiment 1, wherein the accepting of feedback is implemented by processing electrical signals that represent measurements of the subject's nerve or muscle activity.

Embodiment 6 is the method of embodiment 1, wherein the accepting of feedback is implemented by processing electrical signals that represent measurements of electromyography signals to measure muscle activity.

Embodiment 7 is the method of embodiment 1, wherein the accepting of feedback is implemented by processing electrical signals that represent measurements of an accelerometer to measure muscle activity.

Embodiment 8 is a method of ameliorating electrosensation in a subject that is being treated using alternating electric fields, the method comprising: selectively reducing current that flows through one or more different electrode elements during respective different periods of time while alternating electric fields are being applied; accepting feedback that indicates whether electrosensation is occurring during each of the respective different periods of time; and determining whether reducing the current that flows through a given one or more of the electrode elements ameliorates the electrosensation.

Embodiment 9 is the method of embodiment 8, further comprising treating the subject using alternating electric fields while the given one or more electrode elements is operating at a lower current than the other electrode elements.

Embodiment 10 is the method of embodiment 8, wherein the accepting of feedback is implemented by accepting input from the subject.

Embodiment 11 is the method of embodiment 8, wherein the accepting of feedback is implemented by processing electrical signals that represent activity of the subject's nerves from a set of ECAP electrodes.

Embodiment 12 is the method of embodiment 8, wherein the accepting of feedback is implemented by processing electrical signals that represent measurements of the subject's nerve or muscle activity.

Embodiment 13 is the method of embodiment 8, wherein the accepting of feedback is implemented by processing electrical signals that represent measurements of electromyography signals to measure muscle activity.

Embodiment 14 is the method of embodiment 8, wherein the accepting of feedback is implemented by processing electrical signals that represent measurements of an accelerometer to measure muscle activity.

Embodiment 15 is a method of applying electrical signals to a first set of at least four first electrode elements and a second set of at least four second electrode elements positioned on opposite sides of a target region of a subject's body, the method comprising: (a) applying an AC signal between the second set of at least four second electrode elements and a majority of the first electrode elements, wherein one or more different members of the first set of at least four electrode elements are either not used or operate using a reduced current during respective different periods of time; accepting first feedback indicating whether the subject is experiencing electrosensation or is about to experience electrosensation during the respective different periods of time; and determining, based at least in part on the accepted first feedback, whether an amelioration of electrosensation occurs when one or more given members of the first set of at least four electrode elements is either not used or operates using the reduced current.

Embodiment 16 is the method of embodiment 15, further comprising using the second set of at least four second electrode elements and all of the first electrode elements except for the one or more given members of the first set of at least four electrode elements to apply alternating electric fields to the target region.

Embodiment 17 is the method of embodiment 15, further comprising applying alternating electric fields to the target region with the one or more given members of the first set of at least four electrode elements operating at a lower current than other members of the first set.

Embodiment 18 is the method of embodiment 15, wherein the accepting of the first feedback is implemented by accepting input from the subject.

Embodiment 19 is the method of embodiment 15, wherein the accepting of the first feedback is implemented by processing electrical signals that represent activity of the subject's nerves from a set of ECAP electrodes.

Embodiment 20 is the method of embodiment 15, further comprising controlling an amplitude of the AC signal.

Embodiment 21 is the method of embodiment 20, further comprising increasing the amplitude of the AC signal until the determining indicates that the subject is experiencing electrosensation or is about to experience electrosensation.

Embodiment 22 is the method of embodiment 15, further comprising, prior to step (a), applying an AC signal between the second set of at least four second electrode elements and all of the first electrode elements during a first time period; and accepting feedback indicating whether the subject is experiencing electrosensation or is about to experience electrosensation during the first time period.

Embodiment 23 is the method of embodiment 15, further comprising: applying a second AC signal between the first set of at least four first electrode elements and a majority of the second electrode elements, wherein one or more different members of the second set of at least four electrode elements are either not used or operate using a reduced current during respective different periods of time; accepting second feedback indicating whether the subject is experiencing electrosensation or is about to experience electrosensation during the applying of the second AC signal; and determining, based at least in part on the accepted second feedback, whether an amelioration of electrosensation occurs when one or more given members of the second set of at least four electrode elements is either not used or operates using the reduced current.

Embodiment 24 is the method of embodiment 23, further comprising using the first set of at least four first electrode elements and all of the second electrode elements except for the given member of the second set of at least four electrode elements to apply alternating electric fields to the target region.

Embodiment 25 is the method of embodiment 15, wherein the accepting of the first feedback is implemented by processing electrical signals that represent measurements of the subject's nerve or muscle activity.

Embodiment 26 is the method of embodiment 15, wherein the accepting of the first feedback is implemented by processing electrical signals that represent measurements of electromyography signals to measure muscle activity.

Embodiment 27 is the method of embodiment 15, wherein the accepting of the first feedback is implemented by processing electrical signals that represent measurements of an accelerometer to measure muscle activity.

Embodiment 28 is the method of embodiment 15, wherein during the applying, at least two different members of the first set of at least four first electrode elements are either not used or operate using a reduced current during the respective different periods of time, and wherein the determining comprises determining whether an amelioration of electrosensation occurs when at least two given members of the first set of at least four first electrode elements is either not used or operates using the reduced current

Embodiment 29 is an apparatus for applying electrical signals to a first set of at least four first electrode elements and a second set of at least four second electrode elements positioned on opposite sides of a subject's body, the apparatus comprising: an AC signal source having at least one amplitude-control input; a first set of at least four first electrically controlled switches, wherein each of the first switches is configured to, depending on a state of a respective first control input, either (i) close so that current can flow between the AC signal source and a respective first electrode element or (ii) open so that current does not flow between the AC signal source and the respective first electrode element; and a controller configured to control the state of the first control input of each of the first switches. The controller is further configured to (a) apply first control signals to the first control inputs that cause selected ones of the first switches to open at respective periods of time, (b) accept a plurality of first feedback signals indicating whether the subject is experiencing electrosensation or is about to experience electrosensation while the selected ones of the first switches is open at the respective periods of time, and (c) determine, based at least in part on the accepted plurality of first feedback signals, whether an amelioration of electrosensation occurs when a given one of the first switches is open.

Embodiment 30 is the apparatus of embodiment 29, wherein the controller is further configured to control the application of the electrical signals to the first set of at least four electrode elements and the second set of at least four electrode elements so that while the subject is being treated with alternating electric fields, the given one of the first switches is open.

Embodiment 31 is the apparatus of embodiment 29, further comprising a user interface, wherein the user interface is configured to, based on inputs received from the subject, generate the plurality of first feedback signals.

Embodiment 32 is the apparatus of embodiment 29, further comprising an ECAP measurement system configured to accept signals that represent nerve activity from a set of ECAP electrodes, wherein the ECAP measurement system generates the plurality of first feedback signals.

Embodiment 33 is the apparatus of embodiment 29, wherein the controller is further configured to control the amplitude-control input.

Embodiment 34 is the apparatus of embodiment 33, wherein the controller is further configured to, prior to step (a), apply first control signals to the first control inputs that cause all the first switches to close, control the amplitude-control input so that the amplitude increases, and accept a third signal indicating whether the subject is experiencing electrosensation or is about to experience electrosensation.

Embodiment 35 is the apparatus of embodiment 29, further comprising: a second set of at least four second electrically controlled switches, wherein each of the second switches is configured to, depending on a state of a respective second control input, either (i) close so that current can flow between the AC signal source and a respective second electrode element or (ii) open so that current does not flow between the AC signal source and the respective second electrode element, wherein the controller is further configured to control the state of the second control input of each of the second switches. The controller is further configured to (x) apply second control signals to the second control inputs that cause selected ones of the second switches to open at respective periods of time, (y) accept a plurality of second feedback signals indicating whether the subject is experiencing electrosensation or is about to experience electrosensation while the selected ones of the second switches is open at the respective periods of time, and (z) determine, based at least in part on the accepted plurality of second feedback signals, whether an amelioration of electrosensation occurs when a given one of the second switches is open.

Embodiment 36 is the apparatus of embodiment 35, wherein the controller is further configured to control the application of the electrical signals to the first set of at least four electrode elements and the second set of at least four electrode elements so that while the subject is being treated with alternating electric fields, the given one of the second switches is open.

Embodiment 37 is the apparatus of embodiment 35, further comprising a user interface, wherein the user interface is configured to, based on inputs received from the subject, generate the plurality of first feedback signals and the plurality of second feedback signals.

Embodiment 38 is the apparatus of embodiment 35, further comprising an ECAP measurement system configured to accept signals that represent nerve activity from a set of ECAP electrodes, wherein the ECAP measurement system generates the plurality of first feedback signals and the plurality of second feedback signals.

Embodiment 39 is the apparatus of embodiment 35, wherein the controller is further configured to control the amplitude-control input.

Embodiment 40 is the apparatus of embodiment 39, wherein the controller is further configured to, prior to step (a), apply first control signals to the first control inputs that cause all the first switches to close, control the amplitude-control input so that the amplitude increases, and accept a third signal indicating whether the subject is experiencing electrosensation or is about to experience electrosensation.

Headings are provided for convenience only and are not to be construed to limit the invention in any manner. Embodiments illustrated under any heading or in any portion of the disclosure may be combined with embodiments illustrated under the same or any other heading or other portion of the disclosure. Any combination of the elements described herein in all possible variations thereof is encompassed by the invention unless otherwise indicated herein or otherwise clearly contradicted by context.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

1. A method of ameliorating electrosensation in a subject that is being treated using alternating electric fields, the method comprising:
selectively deactivating one or more different electrode elements during respective different periods of time while alternating electric fields are being applied;
accepting feedback that indicates whether electrosensation is occurring during each of the respective different periods of time; and
determining whether deactivating a given one or more of the electrode elements ameliorates the electrosensation.

2. The method of claim 1, further comprising treating the subject using alternating electric fields while the given one or more electrode elements is deactivated.

3. The method of claim 1, wherein the accepting of feedback is implemented by accepting input from the subject.

4. The method of claim 1, wherein the accepting of feedback is implemented by processing electrical signals that represent activity of the subject's nerves from a set of ECAP electrodes.

5.-7. (canceled)

8. A method of ameliorating electrosensation in a subject that is being treated using alternating electric fields, the method comprising:
selectively reducing current that flows through one or more different electrode elements during respective different periods of time while alternating electric fields are being applied;
accepting feedback that indicates whether electrosensation is occurring during each of the respective different periods of time; and
determining whether reducing the current that flows through a given one or more of the electrode elements ameliorates the electrosensation.

9. The method of claim 8, further comprising treating the subject using alternating electric fields while the given one or more electrode elements is operating at a lower current than the other electrode elements.

10. The method of claim 8, wherein the accepting of feedback is implemented by accepting input from the subject.

11. The method of claim 8, wherein the accepting of feedback is implemented by processing electrical signals that represent activity of the subject's nerves from a set of ECAP electrodes.

12.-14. (canceled)

15. A method of applying electrical signals to a first set of at least four first electrode elements and a second set of at least four second electrode elements positioned on opposite sides of a target region of a subject's body, the method comprising:
(a) applying an AC signal between the second set of at least four second electrode elements and a majority of the first electrode elements, wherein one or more different members of the first set of at least four first electrode elements are either not used or operate using a reduced current during respective different periods of time;
accepting first feedback indicating whether the subject is experiencing electrosensation or is about to experience electrosensation during the respective different periods of time; and
determining, based at least in part on the accepted first feedback, whether an amelioration of electrosensation occurs when one or more given members of the first set of at least four first electrode elements is either not used or operates using the reduced current.

16. The method of claim 15, further comprising using the second set of at least four second electrode elements and all of the first electrode elements except for the one or more given members of the first set of at least four first electrode elements to apply alternating electric fields to the target region.

17. The method of claim 15, further comprising applying alternating electric fields to the target region with the one or more given members of the first set of at least four first electrode elements operating at a lower current than other members of the first set.

18. The method of claim 15, wherein the accepting of the first feedback is implemented by accepting input from the subject.

19. The method of claim 15, wherein the accepting of the first feedback is implemented by processing electrical signals that represent activity of the subject's nerves from a set of ECAP electrodes.

20. The method of claim 15, further comprising controlling an amplitude of the AC signal.

21.-28. (canceled)

29. An apparatus for applying electrical signals to a first set of at least four first electrode elements and a second set of at least four second electrode elements positioned on opposite sides of a subject's body, the apparatus comprising:
an AC signal source having at least one amplitude-control input;
a first set of at least four first electrically controlled switches, wherein each of the first switches is configured to, depending on a state of a respective first control input, either (i) close so that current can flow between the AC signal source and a respective first electrode element or (ii) open so that current does not flow between the AC signal source and the respective first electrode element; and
a controller configured to control the state of the first control input of each of the first switches,
wherein the controller is further configured to
(a) apply first control signals to the first control inputs that cause selected ones of the first switches to open at respective periods of time,
(b) accept a plurality of first feedback signals indicating whether the subject is experiencing electrosensation or is about to experience electrosensation while the selected ones of the first switches is open at the respective periods of time, and
(c) determine, based at least in part on the accepted plurality of first feedback signals, whether an amelioration of electrosensation occurs when a given one of the first switches is open.

30. The apparatus of claim 29, wherein the controller is further configured to control the application of the electrical signals to the first set of at least four first electrode elements and the second set of at least four second electrode elements so that while the subject is being treated with alternating electric fields, the given one of the first switches is open.

31. The apparatus of claim 29, further comprising a user interface, wherein the user interface is configured to, based on inputs received from the subject, generate the plurality of first feedback signals.

32. The apparatus of claim 29, further comprising an ECAP measurement system configured to accept signals that represent nerve activity from a set of ECAP electrodes, wherein the ECAP measurement system generates the plurality of first feedback signals.

33. The apparatus of claim 29, wherein the controller is further configured to control the amplitude-control input.

34. The apparatus of claim 33, wherein the controller is further configured to, prior to step (a),
apply first control signals to the first control inputs that cause all the first switches to close,
control the amplitude-control input so that the amplitude increases, and
accept a third signal indicating whether the subject is experiencing electrosensation or is about to experience electrosensation.

35.-40. (canceled)
